(19) United States
(12) Patent Application Publication
(10) Pub. No.: US 2026/0242317 A1
(43) Pub. Date: Aug. 20, 2026

(54) MANUFACTURING 2,3,5-TRIMETHYLHYDROQUINONE FROM A MIXTURE OF MESITOL AND 2,3,6-TRIMETHYLPHENOL

(71) Applicant: DSM IP Assets B.V., Maastricht (NL)

(72) Inventors: Thomas BALDINGER, Kaiseraugst (CH); Werner BONRATH, Kaiseraugst (CH); Thomas BUCHHOLZ, Basel (CH); Alissa GOETZINGER, Kaiseraugst (CH); Ulla LETINOIS, Kaiseraugst (CH); Jonathan Alan MEDLOCK, Kaiseraugst (CH); Dragan MILADINOV, Basel (CH); Jan SCHUETZ, Kaiseraugst (CH); Christof SPARR, Basel (CH); Joël WELLAUER, Basel (CH)

(21) Appl. No.: 19/159,490

(22) PCT Filed: Mar. 1, 2024
(86) PCT No.: PCT/EP2024/055422

§ 371 Date: Aug. 25, 2025

(30) Foreign Application Priority Data
Mar. 1, 2023 (EP) 23159421.9

(51) Int. Cl. C07C 37/01; B01J 31/24; B01J 31/30; C07D 311/72

(52) U.S. Cl. CPC C07C 37/01; B01J 31/2404; B01J 31/30; C07D 311/72; B01J 2231/326; B01J 2531/004; B01J 2531/828

(57) ABSTRACT

The present invention relates to a process of manufacturing 2,3,5-tri-methylhydroquinone from a mixture of mesitol and 2,3,6-trimethylphenol (=2,3,6-TMP). This process offers a highly interesting and commercial interesting way of producing α-tocopherol.

### TECHNICAL FIELD

The present invention relates to the field of manufacturing 2,3,5-trimethylhydroquinone and α-tocopherols.

### BACKGROUND OF THE INVENTION

2,3,5-Trimethylhydroquinone (TMHQ) is a key substance in the synthesis of α-tocopherols. It is typically produced from m-cresol, followed by methylation to yield 2,3,6-trimethylphenol and oxidized to trimethylquinone (TMQ) and reduced to yield TMHQ as disclosed for example by W. Bonrath et al. Angew. Chem. Int. Ed. 2012, 51, 12982-12985 or Bonrath, W. et al. (2021). Vitamins, 4. Vitamin E (Tocopherols, Tocotrienols). In Ullmann's Encyclopedia of Industrial Chemistry. https://doi.org/10.1002/14356007.o27 o07.pub2.

Traditionally, cresols can be extracted from coal tar. However, this leads to a mixture of the isomers o-cresol, m-cresol and p-cresol. From this mixture, o-cresol is easily to be separated, however, m-cresol is very difficult to be isolated from the residual mixture of m-cresol and p-cresol. Therefore, in the current industrial production of TMHQ, m-cresol is sourced by other synthetic pathways leading specifically to the m-isomer. These processes, however, are very complex and expensive which have a high impact on the price of m-cresol suitable for this process.

In the light of increased awareness of sustainability for chemical processes, the interest in using raw material from natural resources has significantly increased.

WO 2015/110654 A1 or WO 2015/110655 A1 have disclosed that 2,5-dimethylphenol or 2,3,6-trimethylphenol, respectively, can be obtained from 2,5-dimethylfuran and ethyne or propyne, respectively, in the presence of Au(I) complexes. However, these procedures lead to isomeric mixtures of phenols. It is known that particularly in the case of ethyne considerable amounts of 2,4-dimethylphenol are produced as side product in the synthesis of the targeted 2,5-dimethylphenol.

New oxidation methods leading to TMHQ were recently found. WO 2021/234077 A1 has disclosed that 2,3,5-trimethylphenol can be photooxidized to 2,3,5-trimethylbenzoquinone.

WO 2022/128852 A1 has disclosed that 2,4,6-trimethylphenol can be photooxidized to 4-hydroperoxy-2,4,6-trimethylcyclohexa-2,5-dien-1-one which can be transformed to 2,3,5-trimethylhydroquinone.

All these documents of the state of the art have in common that the respective syntheses start from specific isomers of dimethylphenol or trimethyl-phenols.

CH 576 928 discloses a process of TMHQ staring from a mixture of 2,3,6- and 2,4,6-trimethylphenol using sulfonation and separation the desired isomer from the undesired isomer. This process, however, is very disadvantageous as only one isomer of trimethylphenol (2,3,6) out of an isomeric mixture is used for the synthesis of the desired 2,3,5-trimethylhydroquinone, whereas the other isomer (2,4,6-TMP) being present in significant amounts (24%) is waste.

### SUMMARY OF THE INVENTION

It was surprisingly found that 2,3,5 trimethylhydroquinone can be formed from a mixture of 2,4,6-trimethylphenol and 2,3,6-trimethylphenol using a process according to claim 1.

This has offered new pathways of using raw materials which are isomeric mixtures of phenols. There is no need for separating said isomers by very costly separation techniques or using complex and expensive synthetic pathways leading to specifically only one isomer for the starting material. It was particularly found that a mixture of m- and p-cresol can be used to yield TMHQ with high yield and selectivity. Furthermore, this invention increases the attractivity of using 2-5-dimethylfuran as a sustainable starting material for the synthesis of TMHQ.

This process is very advantageous in that both isomers 2,4,6-trimethylphenol and 2,3,6-trimethylphenol can be converted to the desired 2,3,5-trimethylhydroquinone.

Further aspects of the invention are subject of further independent claims. Particularly preferred embodiments are subject of dependent claims.

### DETAILED DESCRIPTION OF THE INVENTION

In a first aspect the present invention relates to a process for manufacturing 2,3,5-trimethylhydroquinone (TMHQ) of the formula (I) from a mixture of mesitol (=2,4,6-trimethylphenol=2,4,6-TMP) of the formula (IIa) and of 2,3,6-trimethylphenol (=2,3,6-TMP) of the formula (IIb).

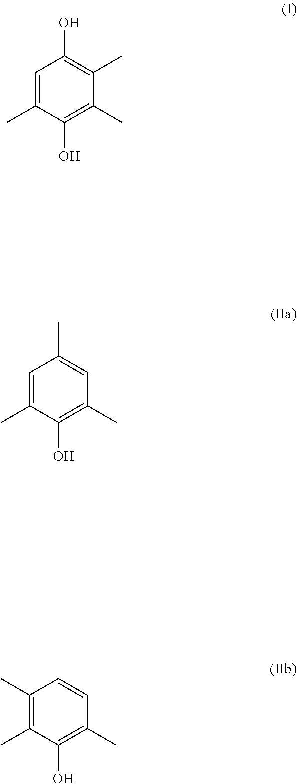

This process comprises the consecutive steps
a) providing a mixture of the compound of the formula (IIa) and the compound of the formula (IIb),

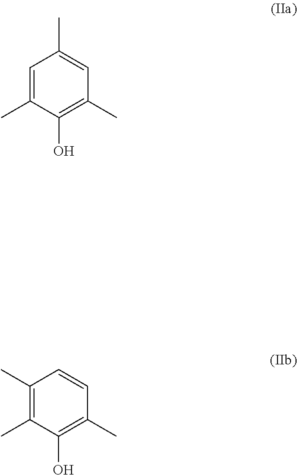

b) oxidizing the mixture of formula (IIa) and the compound of the formula (IIb) to form a mixture of the compound of the formula (IIIa) and of the compound of the formula (IIIb),

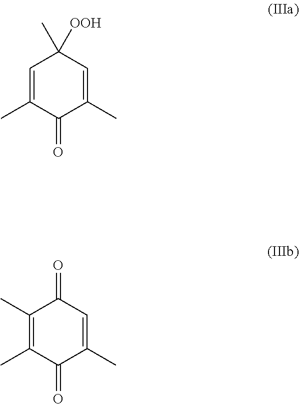

c) reducing the mixture of the compound of the formula (IIIa) and of the compound of the formula (IIIb) by means of a reduction agent to yield a mixture of the compound of the formula (IV) and of the compound of the formula (I),

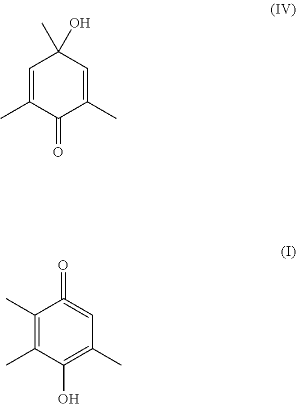

d) rearranging the compound of the formula (IV) in a mixture of the compound of the formula (IV) and of the compound of the formula (I) by treatment of said mixture with a basic substance at a temperature of >200° C., preferably >240° C., to yield the compound of the formula (I)

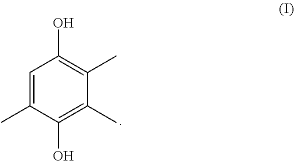

In the present document, a “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH)—CH₂—CH₃ is considered as a C₄-alkyl group.

Analogously, a C_{x-y} alkanol, respectively a C_{x-y} alkylene diol, is an alcohol having one, respectively two, OH groups where the alcohol has an alkyl respectively alkylene group comprising x to y carbon atoms.

The term “independently from each other” in this document means, in the context of substituents, moieties, or groups, that identically designated substituents, moieties, or groups can occur simultaneously with a different meaning in the same molecule.

In case identical labels for symbols or groups are present in several formulae, in the present document, the definition of said group or symbol made in the context of one specific formula applies also to other formulae which comprises the same said label.

The term “inert”, as used in this document in describing a material, means that under the conditions of the reaction said material does not undergo any chemical reaction.

The peak wavelength is the wavelength where the spectrum reaches its highest intensity.

### Providing a Mixture of Mesitol (Formula (IIa)) and 2,3,6-TMP (Formula (IIb))

### (Step a))

In step a) a mixture of the compound of the formula (IIa) (mesitol (=2,4,6-trimethylphenol=2,4,6-TMP) and the compound of the formula (IIb) (of 2,3,6-trimethylphenol (=2,3,6-TMP)) is provided.

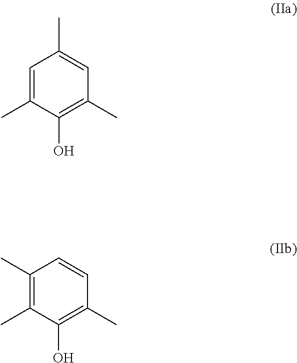

In a first preferred embodiment, said mixture is provided by a methylation step a″) of a mixture of m-cresol and p-cresol to a mixture of mesitol and of 2,3,6-TMP.

Hence, it is preferred that the mixture of the compound (IIa) and the compound of the formula (IIb) is obtained by the reaction step a″)
a″) methylation of a mixture of p-cresol of the formula (0p) and m-cresol of the formula (0m)

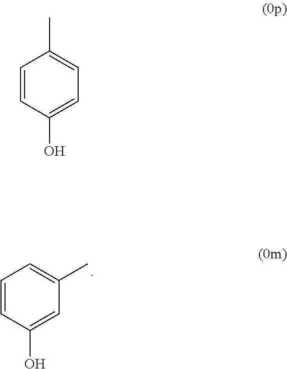

to yield the mixture of the compound of the formula (IIa) and of the formula (IIb).

The methylation in step a″) can be performed by various methods.

In a preferred manner, the mixture of p-cresol and m-cresol is methylated for example in an autoclave with methanol in the presence of lithium hydroxide monohydrate at elevated temperatures as disclosed in EP 1 108 705 A1, particularly by example 3 to yield a mixture of mesitol and 2,4,6-TMP, the whole disclosure of which is incorporated herein by reference.

In an even more preferred manner, the methylation of the mixture of p-cresol and m-cresol is achieved by gas phase methylation, particularly by subjecting the mixture of p-cresol and m-cresol to a mixture of methanol and, optionally, water in the presence of an oxidic catalyst in inert atmosphere at a temperature of between 30° and 500° C., to yield a mixture of mesitol and 2,4,6-TMP. This process produces less of waste material, allows a continuous process and is, therefore, economically and ecologically highly advantageous.

In a second, more preferred embodiment, the mixture of mesitol and 2,3,6-TMP is provided by a methylation step a′) of a mixture of 2,4-dimethylphenol (=2,4-DMP,Formula (IIa-1H)) and 2,5-dimethylphenol (=2,5-DMP,Formula (IIb-H)).

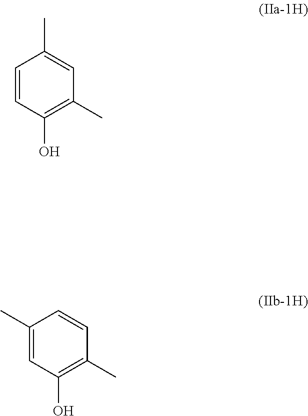

The methylation in step a′) can be performed by various methods.

In a preferred manner, the mixture of 2,4-DMP and 2,5-DMP is methylated example in an autoclave with methanol in the presence of lithium hydroxide monohydrate at elevated temperatures as disclosed in EP 1 108 705 A1, particularly by example 3 to yield a mixture of mesitol and 2,4,6-TMP, the whole disclosure of which is incorporated herein by reference.

In an even more preferred manner, the methylation of the mixture of 2,4-DMP and 2,5-DMP is achieved by gas phase methylation, particularly by subjecting the mixture of 2,4-DMP and 2,5-DMP to a mixture of methanol and, optionally, water in the presence of an oxidic catalyst in inert atmosphere at a temperature of between 30° and 500° C., to yield a mixture of mesitol and 2,4,6-TMP.

Main advantages of the gas phase methylation process are high selectivity resulting in reduced formation of waste and in a continuous process. Using this process leads to particularly high economic and ecological advantages.

The mixture of 2,4-DMP (Formula (IIa-1H)) and 2,5-DMP (Formula (IIb-H)) can be obtained from the reaction of the compound of the formula (V) and ethyne in the presence of a Pt or an Au catalyst, which is either in the form a salt or of a complex.

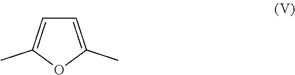

In a third preferred embodiment, the mixture of mesitol and 2,3,6-TMP is obtained from the reaction of the compound of the formula (V) and propyne in the presence of a Pt or an Au catalyst, which is either in the form a salt or of a complex.

Therefore, in a very preferred embodiment, the mixture of the compound (IIa) and the compound of the formula (IIb) is obtained by a reaction step a0)
a0) reacting the compound of the formula (V) with the compound of the formula (VI)

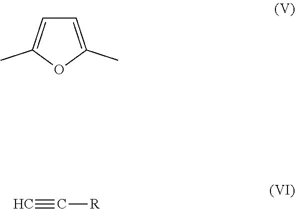

wherein R represents H or CH₃, preferably CH₃;
in the presence of a Pt or an Au catalyst, which is either in the form a salt or of a complex to form a mixture of the compound of the formula (IIa-1) and of the formula (II-b)

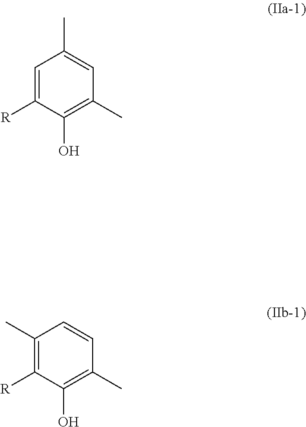

with the proviso that in case R represents H, the mixture of the compound of the formula (IIa-1H) and of formula (IIb-1H) is submitted to a reaction step a′)

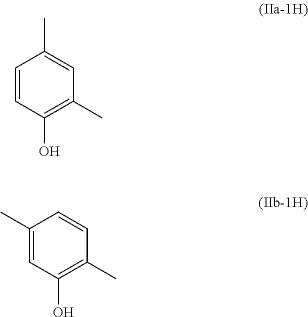

a′) methylation of the mixture of the formula (IIa-1H) and of the formula (IIb-1H) to yield the mixture of the compound of the formula (IIa) and of the formula (IIb)

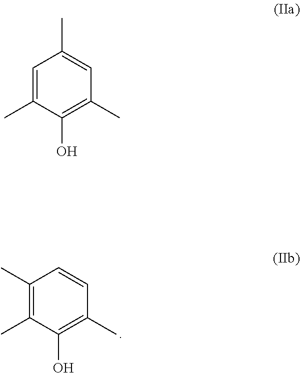

2,5-Dimethylfuran is reacted with propyne (R═CH₃) or acetylene (R═H). Both propyne and acetylene are gases.

In case of R represents CH₃, the propyne can be also mixed with propadiene. A particular mixture comprising propyne and propadiene is known as MAF gas (MAF=methylacetylene-allene fraction) to the person skilled in the art. MAF gas is a cheap process gas and is commercially available from different suppliers.

In one embodiment, the step a0) is performed in the presence of a gold catalyst.

This is described in great details in WO 2015/110655 A1 (using ethyne) or WO 2015/110654 A1 (using propyne), the whole disclosure of which is incorporated herein by reference.

In another embodiment, the step a0) is performed in the presence of a platinum catalyst.

Said platinum catalyst is preferably either in the form of a platinum salt or platinum complex.

In one embodiment said platinum catalyst is preferably a Pt(II) salt, particularly or PtCl₂.

In another embodiment, said platinum catalyst is preferably a complex of Pt(II), particularly a complex of Pt(II) having at least one organic ligand comprising at least one phosphorous atom, particularly a ligand selected from the group consisting of phosphites, phosphates, phosphonates, and phosphines.

Particularly preferred as Pt catalyst is PtCl₂ in the presence of an organic ligand having at least comprising at least one phosphorous atom, particularly a ligand selected from the group consisting of phosphites, phosphates, phosphonates, and phosphines.

Said organic ligand is preferably selected from the group consisting of triphenylphosphite (P(OPh)₃), diphenylphosphate ((PhO)₂P═O(OH)), triphenyl-phosphate ((PhO)₃P═O), triethylphosphate ((EtO)₃P═O), dibenzylphosphite ((PhCH₂O)₂P═O(H)), methyldiphenylphosphite ((MeO)P(OPh)₂), triphenylphosphine (P(Ph)₃), P(Ph_{5F})₃, bis-(2,2,2-trifluorethyl)-phosphite ((CF₃CH₂O)₂P═O(H)), triethyl 2-fluoro-2-phosphonoacetate ((CHFCOOEt)(EtO)₂P═O), tris(2-tolyl)-phosphite, ethyl 3-(diethoxyphosphoryl)propanoate (=triethyl 3-phosphonopropionate) ((CH₂CH₂COOEt)(EtO)₂P═O), 2-(diphenylphosphino)benzoic acid (P(Ph)₂C₆H₄COOH), 3,9-Bis(2,4-di-tert-butylphenoxy)-2,4,8,10-tetraoxa-3,9-diphosphaspiro[5.5]undecane (Ultranox 626), P(O-isooctyl)₃), P(O-isopropyl)₃), P(Oi-PrF₆)₃, P(On-Bu)₃, bis(2,4-di-tert-butylphenoxy)-λ²-phosphane, CH₃P(EtO)₂, 1,1′-binaphthyl-2,2′-diyl hydrogenphosphate (=4-hydroxydinaphtho[2,1-d:1′,2′-f][1,3,2]dioxaphosphepine 4-oxide), VAPOL hydrogen phosphate (=18-hydroxy-8,9-diphenyldiphenanthro[4,3-d:3′,4′-f][1,3,2]dioxaphosphepine 18-oxide), methyltriphenoxyphosphonium iodide ([CH₃P(OPh)₃]⁺[⁻, 4-ethyl-2,6,7-trioxa-1-phosphabicyclo[2.2.2]octane ((CH₂CH₂COOEt)(EtO)₂P═O, trimethylolpropane phosphite), trisodium triphenylphosphine trisulfonate (=TPPTS), disodium triphenylphosphinedisulfonate (=TPPDS) and sodium triphenylphosphine-monosulfonate (=TPPMS);
wherein Ph represents phenyl, Ph_{5F} represents pentafluorophenyl and Et represents ethyl and Me represents methyl.

The organic ligand is more preferably selected from the group consisting of triphenylphosphite (P(OPh)₃),diphenylphosphate ((PhO)₂P═O(OH)), dibenzyl-phosphite ((PhCH₂O)₂P═O(H)), triphenylphosphate ((PhO)₃P═O), methyldiphenyl-phosphite ((MeO)P(OPh)₂), trisodium triphenylphosphine trisulfonate (=TPPTS), P(Ph_{5F})₃, bis-(2,2,2-trifluorethyl)-phosphite ((CF₃CH₂O)₂P═O(H)), tris(2-tolyl)phosphite, ethyl 3-(diethoxyphosphoryl)propanoate (=triethyl 3-phosphonopropionate) ((CH₂CH₂COOEt)(EtO)₂P═O), 2-(diphenylphosphino)benzoic acid (P(Ph)₂C₆H₄COOH) and 9-Bis(2,4-di-tert-butylphenoxy)-2,4,8,10-tetraoxa-3,9-diphosphaspiro[5.5]undecane (Ultranox 626); wherein Ph represents phenyl, Ph_{5F} represents pentafluorophenyl and Et represents ethyl and Me represents methyl.

It is preferred that said reaction between 2,5-dimethylfuran and propyne or acetylene is performed in the presence of an ether or a ketone, particularly a cyclic ether, preferably tetrahydrofuran, or acetone or methyl ethyl ketone or diethyl ketone, preferably acetone.

It is further preferred that the amount of the Pt catalyst is present in an amount in the range of 0.1-25 mol %, particularly 6-12 mol %, in respect to the compound of the formula (V).

It is further preferred that molar ratio of the above-mentioned organic ligand comprising at least one phosphorous atom to Pt is in the range of 1-2, preferably 0.5-1.5, more preferably 0.4-1.2.

It is preferred that the molar ratio of compound of the formula (V) to compound of the formula (VI) is in the range of 1:1 to 1:8, particularly of 1:1 to 1:8, preferably 1:1 to 1:3.

It is further preferred that the reaction is performed at a temperature of between 0° C. and 80°, particularly of between 10° C. and 60° C., preferably of between 20° C. and 30° C.

At temperatures being above the boiling point of the solvent, the reaction is preferably performed under pressure.

The above process produces a mixture of the compound of the formula (IIa) and of the formula (IIb) or of the formula (IIa-1H) and of the formula (IIb-1H), respectively.

In case of R being H, the formation of 2,4-DMP (IIa-1H) is predominantly formed. Typically, the molar ratio of 2,4-DMP: 2,5-DMP is larger than 50:50.

As in the present process also 2,4-DMP, having of a methyl group in the para position to the phenolic OH group in 2,4-DMP, can be used to yield the desired 2,3,5 TMHQ, the formation of a high content in 2,5-DMP is not a disadvantage.

It has been observed that the molar ratio of compound of formula (IIa-1H) (=2,4-dimethylphenol=2,4-DMP): compound of formula (IIb-1H) (=2,5-dimethyl-phenol=2,5-DMP) of typically <70:30, particularly <60:40, preferably <50:50.

In case of R being CH₃, the molar ratio of compound of formula (IIa): compound of formula (IIb) is ≤50:50, particularly <10:90, more particularly <5:95, preferably <3:97, more preferably <2:98.

### Oxidation (Step b))

In the step b) the mixture of compound of mesitol (formula (IIa)) and 2,3,6-TMP (formula (IIb)) is oxidized to form a mixture of the compound of the formula (IIIa) and of the compound of the formula (IIIb) provided.

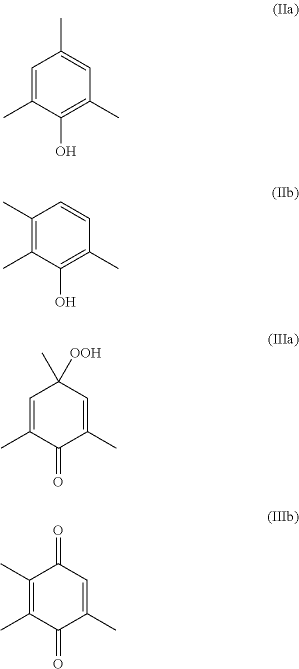

In one of the embodiments, the oxidation of step b) is a classical chemical oxidation and can be performed by methods principally known to the person skilled in the art.

Particularly, it can be performed by molecular oxygen, particularly in the presence of a cobalt complex and/or in the presence of a base, particular an alkali metal salt, details of which are as disclosed in DE 2 314 600 or DE 2 747 497.

Furthermore, the oxidation of step b) can be performed by chlorine in a suitable solvent preferably in the absence of a base, followed by hydrolysis with water as described in U.S. Pat. No. 4,612,401, the whole disclosure of which is incorporated herein by reference.

Furthermore, the oxidation of step b) can be performed by hypohalo-genous acid or salt in an aqueous medium or a mixture of water and an organic solvent; details of which are as disclosed in EP 0 084 158 A1, the whole disclosure of which is incorporated herein by reference.

In another, more preferred, embodiment the oxidation of step b) is a photochemical oxidation.

It is particularly preferred that the oxidation step b) is a photooxidation using oxygen and a photosensitizer of the formula (X)

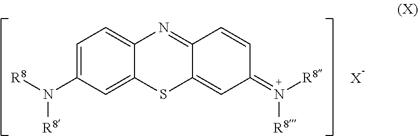

wherein R⁸, R^{8′}, R^{8″} and R^{8′″} independently from each other represent either a H, or a C₁₋₄ alkyl group;
or
wherein R⁸ and R^{8′} and/or R^{8″} and R^{8′″} form together with N a five or six membered ring;
with the proviso that at least one of the residues R⁸, R^{8′}, R^{8″} and R^{8′″} is different from H;
and X⁻ represents an anion;
in a solvent mixture of water and at least one C₁₋₈ alkanol or at least one C₂₋₄ alkylene diol;
and using light which has a peak wavelength (λₘₐₓ) in its spectrum in the range of between 580 and 780 nm.

More details of the preferred photoxidation can be found in WO 2022/128852 A1, the whole disclosure of which is incorporated herein by reference.

In one embodiment, R⁸ and R^{8′} and/or R^{8″} and R^{8′″} form together —(CH₂)₅— or —(CH₂)₂—NH—(CH₂)₂— or —(CH₂)₂—N(C₁₋₄ alkyl)-(CH₂)₂— or —(CH₂)₂—S—(CH₂)₂— or —(CH₂)₂—O—(CH₂)₂—.

It is further preferred that R⁸═R^{8″} and/or R^{8′}═R^{8′″}. More preferred is that R⁸═R^{8′}═R^{8″}═R^{8′″}.

More preferably, the substituent R⁸, R^{8′}, R^{8″} and R^{8′″} represent a C₁₋₄ alkyl group, even more preferably R⁸═R^{8′}═R^{8″}═R^{8′″}=methyl or ethyl.

Most preferably R⁸═R^{8′}═R^{8″}═R^{8′″}═CH₃.

In formula (X) X⁻ represents an anion. The role of the anion is to counter balance the charge of the cation which is represented in the above formula by the part within the brackets ([)(]). Therefore, in principle any anion can be used.

Preferably, X⁻ represents a halide, most preferably a chloride.

Preferably, the compound of the formula (X) is methylene blue. Further preferred is the compound of the formula (X) in the form of a double salt with zinc chloride, particularly a double salt of methylene blue with zinc chloride or in the form of a hydrate, preferably methylene blue hydrate (CAS:122965-43-9).

It is essential that for the above photooxidation light is used which has a peak wavelength (λₘₐₓ) in its spectrum in the range of between 580 and 780 nm.

In one preferred embodiment light is used which has a peak wavelength (λₘₐₓ) in its spectrum in the range of between 585 and 625 nm. This corresponds to a light which is perceived as orange.

In another, more preferred, embodiment light is used which has a peak wavelength (λₘₐₓ) in its spectrum in the range of between 625 and 740 nm. This corresponds to a light which is perceived as red.

This light is mainly of the high wavelength range of the visible spectrum.

In a further preferred embodiment, the light used is characterized so that more than 80% of the light has a wavelength of between 525 and 780 nm, preferably more than 80% of the light has a wavelength of between 525 and 700 nm, more preferably more than 65% of the emitted light has a wavelength of between 550 and 650 nm.

In an even further preferred embodiment, the light used is characterized so that more than 80% of the light has a wavelength of between 550 and 780 nm, preferably more than 80% of the light has a wavelength of between 600 and 760 nm, more preferably more than 65% of the emitted light has a wavelength of between 625 and 700 nm, most preferably more than 85% of the emitted light has a wavelength of between 625 and 700 nm.

It is, therefore, important that the light used has no significant amount of light having a wavelength below 580 nm in its spectrum. It is essential that light of the colours green, blue and violet or colours having significant amounts green, blue and violet in their spectrum have been found not to be suited for the above photooxidation.

In one embodiment, the light which is used for the photooxidation can be realized by filtering the undesired light wavelengths from a light source. For example, a light source having a multichromatic or white emission can be filtered by a filter which blocks off the undesired wavelength.

There are different possibilities of such filters known and commercially available such as absorption, dichroic, monochromatic, band-pass, short-pass or wedge filters, using different physical methods for filtration of light.

Particularly useful are absorption or cut-off filters.

It is particularly preferred that the light source is a white LED lamp in combination with a filter blocking the wavelengths below 500 nm, most particularly below 625 nm.

In a further embodiment, the light which is used for the photooxidation can be produced by a respective light source emitting light of the desired wavelengths. The light source is preferably an orange or red light, more preferably an orange or red LED to provide a light using light which has a peak wavelength (λₘₐₓ) in its spectrum in the range of between 580 and 780 nm.

Specific examples of light sources of this embodiment are red LEDs or red or orange Lasers, preferably red or orange LED lamps. Red and orange LED lamps are commercially broadly available. Red and orange LEDs can provide high intensities of red or orange light. In a preferred embodiments a flexible strip having a plurality of individual LEDs incorporated in said strip. This allows to assure radial orientation of the LED around a curved surface such as a transparent tube, for example, by simply wrapping, preferably in a helical manner, said strip around the tube.

A red LED lamp is the most preferred light source for the light.

The photooxidation is performed in a solvent mixture of water and at least one C₁₋₈ alkanol or at least one C₂₋₄ alkylene diol.

The C₁₋₈ alkanol is preferably selected from the group consisting of methanol, ethanol, propanol, isopropanol, butanol, heptanol and hexanol, more preferably selected from the group consisting of methanol, ethanol and isopropanol.

The C₂₋₄ alkylene diol is preferably selected from the group consisting of ethane-1,2-diol, propane-1,2-diol, propane-1,3-diol, butane-1,3-diol, butane-1,4-diol, butan-1,2-diol and butane-2,3-diol, preferably selected from the group consisting of ethane-1,2-diol, propane-1,2-diol and propane-1,3-diol.

It is preferred that the solvent mixture is a mixture of water and at least one C₁₋₈ alkanol or at least one C₂₋₄ alkylene diol form a homogeneous phase.

It is preferred that the solvent mixture is a mixture of water and at least one C₁₋₈ alkanol or at least one C₂₋₄ alkylene diol. More preferred the solvent mixture is a mixture of water and C₁₋₈ alkanol.

Even more preferred the solvent mixture is a mixture of water and C₁₋₆ alkanol.

More preferably the solvent mixture is a mixture of water and methanol and/or ethanol and/or isopropanol. Most preferably, the solvent mixture is a mixture of water and methanol and/or ethanol.

It is preferred that the volume ratio of water to the sum of C₁₋₈ alkanol and C₂₋₄ alkylene diol is in the range of between 1:10 and 1:1, particularly between 1:5 and 1:2.

In a very preferred embodiment, the solvent mixture is a mixture of water and methanol, preferably in a volume of water to methanol ratio in the range of 1:20 to 1:2, preferably of 1:10 and 1:2, more preferably of 1:6 and 1:3, most preferably 1:4.

It is a key advantage that the photooxidation is made in a solvent mixture consisting of water and at least one C₁₋₈ alkanol or at least one C₂₋₄ alkylene diol, which are ecologically and ecotoxicologically all very favourable solvents and are also economically advantageous. Hence, it is very favourable that the above process is performed in the absence of any chlorinated solvent.

It is preferred that the concentration of the mixture of the compounds of the formulae (IIa) and (IIb) is in the range of between 0.002 to 2.0 mol/l, preferably 0.01 to 0.2 mol/l at the beginning of the photooxidation.

Further preferred is that the ratio of the compound of the formula (X) to the compounds of the formula (IIa) and (IIb) is in the range of between 0.005 and 20 mol %, preferably between 0.05 and 20 mol %, more preferably between 0.2 and 10 mol %.

By the photoreaction a mixture of the compound of the formula (IIIa) and (IIIb) is produced by photochemical reaction from the mixture of the compound (IIa) and (IIb) and oxygen, particular in a gas mixture comprising at least 15% by volume of oxygen.

In one embodiment, oxygen is used in a form of a mixture comprising oxygen and an inert gas. It is preferred that the amount of oxygen in such a mixture comprising oxygen and an inert gas is at least 15% by volume, particularly at least 20% by volume. Such a mixture may, for example, be a binary mixture such as a mixture oxygen/nitrogen or oxygen/argon or alike. Said mixture can consist of or comprise two or more inert gases. It is particularly preferred to use air as such a mixture comprising oxygen and an inert gas.

In a preferred embodiment, oxygen is used in a substantially pure form, i.e. that the amount of oxygen in the gas is 90%-100%, more preferably 95%-100%, even more preferably 99%-100%.

The photooxidation can take place at ambient pressure or under pressure. It is preferred that the oxidation takes place under pressure, particularly under a pressure of more than 2 bar, preferably more than 3 bar, more preferably under a pressure of between 2 and 20 bar.

The photooxidation is performed in a suitable photoreactor. A preferred photoreactor is a flow reactor, particularly in a spiral flow reactor.

The individual components can be introduced separately or as mixture into the photoreactor. Preferably the reaction mixture is prepared before entering into the photoreactor.

The reaction preferably is processed in such a manner that the pressure of oxygen is controlled by suitable valves and mass flow controller. Such process control equipment and methods for photoreactions using liquids and gases is known by the person skilled in the art.

It is preferred that the photooxidation is made in a reactor allowing a continuous process, as it is preferred that said process is a continuous process.

It was observed that the mixture of the compound of the formula (IIIa) (4-hydroperoxy-2,4,6-trimethylcyclohexa-2,5-dien-1-one) and the compound of the formula (IIIb) can be obtained in very high yield, preferably more than 95%, even more preferably more than 98%, and very high selectivity by this photooxidation process (step b)).

### Reduction (Step c))

In step c) the mixture of the compound of the formula(IIIa) and of the compound of the formula (IIIb) are reduced by means of a reduction agent to yield a mixture of the compound of the formula(IV) and of the compound of the formula(I).

For the reduction in step c) several reducing agents can be used.

Suitable as reducing agents can be thiosulphates, tertiary phosphine, hydrogen, dithionates, dithionites, sulfites, trialkylphosphites, iodides, metals or dialkylsulfides.

The reducing agent is preferably selected from the group consisting of Na₂S₂O₃ (sodium thiosulphate), PPh₃, (triphenylphosphine), H₂/PdC, Na₂S₂O₄ (sodium dithionite), Na₂SO₃ (sodium sulfite), P(OEt)₃ (triethyl phosphite), NaI (sodium iodide), Zn (and/or other metals) and DMS (dimethyl sulfide).

Preferred as reducing agents are thiosulphate, particularly sodium thiosulphate.

It is preferred that the reducing agent is used in a significant molecular excess, most preferably in an amount of between 2 to 10 equivalents relative to compound of the formula (I). It is further preferred that the reduction is made in a aqueous alcohol particularly at room temperature.

The reduction can be performed in quantitative scale and very high yields.

The reduction in step c) can be performed in a batch-process or in a continuous process.

It is preferred that the step c) is performed in a continuous way.

### Rearrangement (Step d))

In step d) the compound of the formula (IV), in the mixture of the compound of the formula (IV) and of the compound of the formula (I), is rearranged by treatment of said mixture with a basic substance at a temperature of >200° C., preferably >240° C., to yield the compound of the formula (I).

Usable as such basic substance are particularly the alkali metals such, for example, as sodium, potassium, lithium, rubidium and caesium; the alkaline earth metals such, for example, as calcium, magnesium, barium, and strontium; as well as the basic compounds containing at least one of these metals in their molecular structure. The following compounds are mentioned as examples for such basic substances:
A. The hydroxides of alkali metals or alkaline earth metals such, for example, as sodium hydroxide, potassium hydroxide, lithium hydroxide, calcium hydroxide, magnesium hydroxide and barium hydroxide; and
B. The carbonates and bicarbonates of alkali metals or alkaline earth metals such, for example, as sodium carbonate, sodium bicarbonate, potassium carbonate, potassium bicarbonate, calcium carbonate, barium carbonate and magnesium carbonate; and
C. The oxides of alkaline earth metals such, for example, as calcium oxide, magnesium oxide and barium oxide; and
D. The alkali metal- or alkaline earth metal-containing compounds that have hitherto been used as buffers such, for example, as a suitable mixture of an alkali dihydrogen phosphate such as monopotassium dihydrogen phosphate and a dialkali monohydrogen phosphate such as dipotassium monohydrogen phosphate, or the alkali metal salts of such organic carboxylic acids as boric acid, citric acid, lactic acid, tartaric acid and acetic acid; and
E. Metal oxides, particularly iron oxides.

It is preferred that step d) is performed in the presence of water. It is further preferred that next to water at least one water-soluble alcohol, preferably methanol and/or ethanol and/or iso-propanol, is present in step c).

The step d) is preferably carried out in the presence of the basic substance so that pH is not less than 6.5, and preferably not less than 7. A most preferred pH of the reaction mixture is 7-14.

It is preferred that the step d) is performed under reducing conditions or under inert atmosphere, particularly under nitrogen or argon.

Preferably the basic reaction mixture is neutralized at the end of the reaction by means of an acid.

Preferably, the rearrangement step d) is performed as disclosed in U.S. Pat. No. 3,957,887, particularly as described in its example 12, or in FR 2 200 225 or DE 2 345 062, the whole disclosure of which is incorporated herein by reference.

The reaction of step d) can be performed in a batch-process or in a continuous process.

It is preferred that the step d) is performed in a continuous way.

Reduction/Rearrangement (Step c/d))

In a preferred embodiment, the reducing step c) and the rearranging step d) are performed as a combined single step c/d)
c/d) reducing/rearranging by treatment of the mixture of formula (IIIa) and of the compound of the formula (IIIb) with a basic substance and the presence of a reduction agent at a temperature of >200° C., preferably >240° C., to yield the compound of the formula (I).

The details of the reduction and rearrangement are already described before for the step c) or the step d), respectively.

The present invention shows that 2,3,5-trimethylhydroquinone can be obtained in high yield and selectivity from a mixture of 2,4,6-TMP and 2,3,6-TMP which particularly can be obtained either from 2,5-dimethylfuran being obtained from renewable sources or from mixtures of m- and p-cresol, which, as a mixture, is easily and cheap available from commercial suppliers in large industrial volumes. It is particularly surprising that both components of the respective mixtures can undergo the reaction at the same conditions and undisturbed from each other at each of the respective reaction steps.

As already mentioned, 2,3,5-trimethylhydroquinone is key ingredient in the synthesis of α-tocopherol.

Hence, the present invention relates also to the process of manufacturing α-tocopherol comprising the steps
i) providing 2,3,5-trimethylhydroquinone manufactured from a mixture of mesitol (formula (IIa)) and 2,3,6-TMP (formula (IIb)) by a process as discussed above in great details;
ii) condensing 2,3,5-trimethylhydroquinone from step i) with a compound of the formula (VII-A) or of the formula (VII-B) to yield α-tocopherol of the formula (VIII)

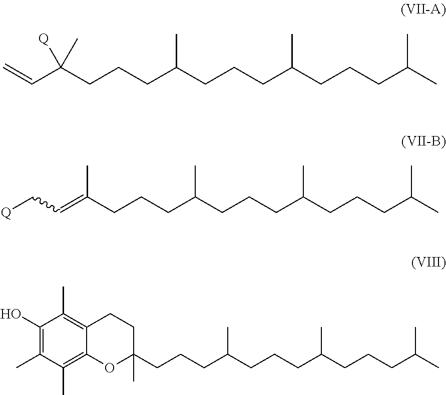

wherein Q is a substituent selected from the group consisting of OH, halide, acyloxy, particularly acetoxy or benzoyloxy, methanesulfonyloxy (=mesyloxy), ethanesulfonyloxy, benzenesulfonyloxy and toluenesulfonyloxy (=tosyloxy);
wherein any wavy line represents a carbon-carbon bond which when linked to the carbon-carbon double bond is either in the Z- or in the E-configuration.

In case Q represents a halide, preferably Q represents Cl.

In case Q represents an acyloxy the acyloxy is preferably a group of the formula

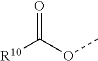

wherein R¹⁰ represents either an C₁₋₆-alkyl or an aryl group, which is optionally substituted, particularly by at least one C₁₋₆-alkyl group.

Preferably R¹⁰ represents either an C₁₋₆-alkyl or to a phenyl group.

More preferably, R¹⁰ represents either a methyl or a phenyl group, most preferably a methyl group.

Particular examples for compounds of formula (VII-A) are isophytol, isophytyl chloride, isophytyl bromide, isophytyl iodide, isophytyl acetate, isophytyl methanesulfonate, isophytyl ethanesulfonate, isophytyl benzenesulfonate, and isophytyl toluenesulfonate.

Particular examples for compounds of formula (VII-B) are phytol, phytyl chloride, phytyl bromide, phytyl iodide, phytyl acetate, phytyl methanesulfonate, phytyl ethanesulfonate, phytyl benzenesulfonate, and phytyl toluenesulfonate.

The compound of formula (VII-B) can be used as E/Z-mixture as well as in pure E- or pure Z-form. Preferred is their use as E/Z-mixtures.

Q represents preferably OH or Cl.

Hence, preferred as compound of formula (VII-A) or (VII-B) are phytol, isophytol, phytyl chloride or isophytyl chloride, more preferred phytol or isophytol.

Most preferred is isophytol.

The use of compounds of formula (VII-A) are preferred over compounds of formula (VII-B).

The condensation step ii) is schematically shows in FIG. 3.

It has been found that the condensation reaction of step ii) can be performed as described for example in W. Bonrath et al. Angew. Chem. Int. Ed. 2012, 51, 12982-12985 or Bonrath, W. et al. (2021). Vitamins, 4. Vitamin E (Tocopherols, Tocotrienols). In Ullmann's Encyclopedia of Industrial Chemistry. https://doi.org/10.1002/14356007.o27 o07.pub2.

This condensation reaction (step ii)) is preferably performed using a Lewis or a Brønsted acid.

Said Lewis or a Brønsted acid are particularly those as mentioned in EP 0949255 A1 and Bonrath et al., Adv. Synth. Catal. 2002, 344, 37-39.

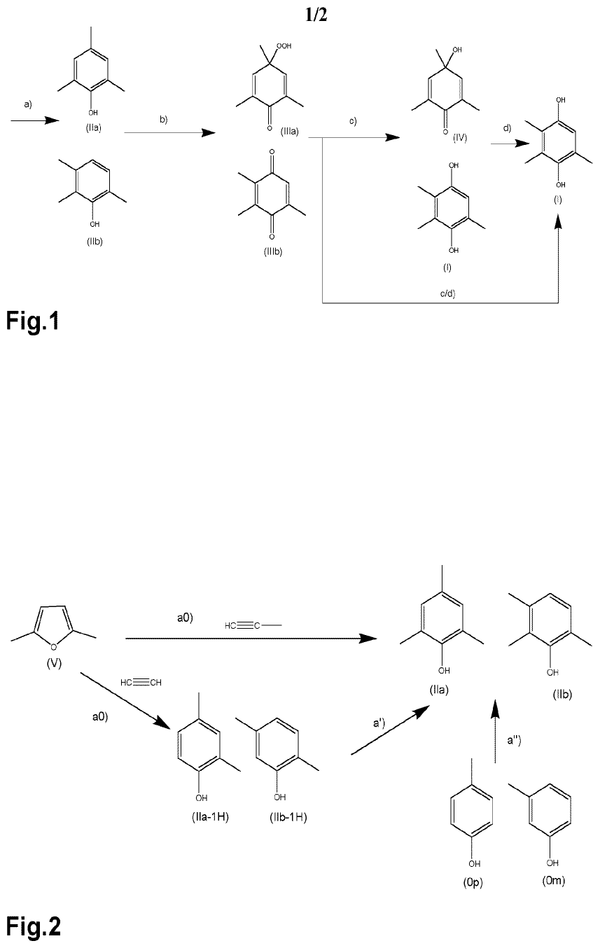

FIG. 1 schematically show the different preferred synthetic pathways of the process of manufacturing 2,3,5-trimethylhydroquinone of the formula (I) from a mixture of mesitol (formula IIa) and 2,3,6-TMP (formula IIb) as discussed above in great details.

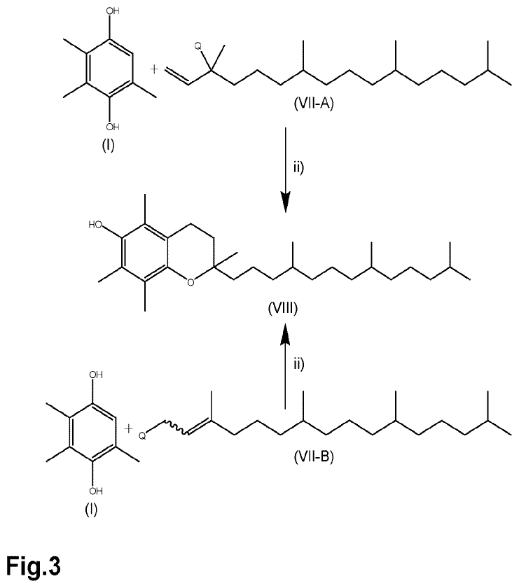

FIG. 2 schematically show the different preferred synthetic pathways to yield a mixture of mesitol (formula IIa) and 2,3,6-TMP (formula IIb) as discussed above in great details.

FIG. 3 schematically show the manufacturing of alpha-tocopherol (formula (VIII)) from 2,3,5-trimethylhydroquinone of the formula (I).

### Examples

The present invention is further illustrated by the following experiments.

First Experimental Series (Examples 1-26): Reaction of 2,5-Dimethylfuran with Acetylene: Step a0)

2,5-Dimethylfuran was reacted with acetylene (2% by weight) in an organic solvent and a platinum catalyst as indicated in table 1.

For this, the platinum chloride (6 mol %), 2,5-dimethylfuran (1.0 equivalent) and acetylene (1.0 equivalent) dissolved (2% by weight) in the respective organic solvent were added to a 10 ml vial with a magnetic stirrer bar under argon atmosphere. The vial was then sealed with a cap and the mixture was stirred in an aluminium block at 23° C. during a time as indicated in table 1. The reaction mixture was then filtered to remove the catalyst and ligand. All volatiles (including non-reacted 2,5-dimethylfuran) were removed in vacuo at 50° C./<30 mbar to give an oily residue. The amounts of 2,4- and 2,5-dimethylphenol were determined by GC (gas chromatography). The ratio as well as the yield are indicated in table 1.

Results of table 1 show that particularly the ligands having an aromatic substituent are suitable as part of the platinum catalyst. The ligand P(Ph_{5F})₃ (tris(pentafluoro-phenyl)phosphine) is one of the most suitable ligands. Furthermore, table 1 shows that acetone and 3-pentanone are particularly well suited as organic solvents. All examples of table 1 yield a mixture of 2,4 DMP and 2,5 DMP.

TABLE 1
Reaction of 2,5-dimethylfuran with acetylene in different solvents and different catalysts

| | | | Ligand | | Yield | |
|---|---|---|---|---|---|---|
| Ex. | Acetylene¹ | Ligand | [mol %]² | Time [h] | [wt %]³ | 2,4:2,5⁴ |
| 1 | acetone | P(OPh)₃ | 6 | 120 | 25 | 65:35 |
| 2 | acetone | P(OPh)₃ | 12 | 148 | 32 | 65:35 |
| 3 | THF | P(OPh)₃ | 12 | 148 | 5 | 68:32 |
| 4 | acetone | (PhO)₂P═O(OH) | 6 | 148 | 12 | 68:32 |
| 5 | acetone | (PhCH₂O)₂P═O(H) | 6 | 148 | 22 | 61:39 |
| 6 | acetone | (PhCH₂O)₂P═O(H) | 6 | 148 | 21 | 59:41 |
| 7 | THF | (PhCH₂O)₂P═O(H) | 6 | 148 | 6 | 66:34 |
| 8 | acetone | (PhO)₃P═O | 6 | 148 | 11 | 43:57 |
| 9 | acetone | (MeO)P(OPh)₂ | 6 | 148 | 27 | 60:40 |
| 10 | acetone | (MeO)P(OPh)₂ | 12 | 148 | 36 | 62:38 |
| 11 | THF | (MeO)P(OPh)₂ | 6 | 148 | 7 | 68:32 |
| 12 | acetone | TPPTS | 6 | 148 | 13 | 57:43 |
| 13 | THF | TPPTS | 6 | 148 | 8 | 56:44 |
| 14 | acetone | P(Ph₅F)₃ | 6 | 148 | 45 | 66:34 |
| 15 | acetone | (CF₃CH₂O)₂P═O(H) | 6 | 148 | 10 | 56:44 |
| 16 | acetone | (CHFCOOEt)(EtO)₂P═O | 6 | 148 | 13 | 52:48 |
| 17 | acetone | tris(2-tolyl)phosphite | 6 | 148 | 14 | 58:42 |
| 18 | acetone | (CH₂CH₂COOEt)(EtO)₂P═O | 6 | 148 | 6 | 48:52 |
| 19 | acetone | P(Ph)₂C₆H₄COOH | 6 | 148 | 6 | 61:39 |
| 20 | acetone | Ultranox 626 | 6 | 148 | 3 | 50:50 |
| 21 | 3-pentanone⁴ | P(OPh)₃ | 6 | 148 | 28 | n.d.⁷ |
| 22 | 3-pentanone⁶ | P(OPh)₃ | 6 | 148 | 21 | n.d.⁷ |
| 23 | 3-pentanone⁶ | P(Ph_{5F})₃ | 6 | 148 | 16 | n.d.⁷ |
| 24 | 3-pentanone⁵ | P(Ph_{5F})₃ | 6 | 148 | 48 | n.d.⁷ |
| 25 | 3-pentanone⁶ | P(Ph_{5F})₃ | 6 | 148 | 33 | n.d.⁷ |
| 26 | 3-pentanone⁶ | P(Ph_{5F})₃ | 6 | 148 | 28 | n.d.⁷ |

¹acetylene 2% in mentioned organic solvent
²relative to 2,5-dimethylfuran
³combined yield of 2,4-DMP and 2,5-DMP
⁴2,4:2,5: ratio 2,4-DMP: 2,5-DMP (area %)
⁵molar ratio acetylene: 2,5-DMF = 2:1
⁶molar ratio acetylene: 2,5-DMF = 3:1
⁶molar ratio acetylene: 2,5-DMF = 5:1
⁷n.d.: not determined

Second experimental series(examples 27-37): Reaction of 2,5-dimethylfuran with propyne: Step a0)

2,5-Dimethylfuran was reacted with propyne (2% by weight) in an organic solvent and a platinum catalyst as indicated in table 2.

For this, the platinum chloride (6 mol %), 2,5-dimethylfuran (1.0 equivalent) and propyne (1.0 equivalent) dissolved (2% by weight) in the respective organic solvent were added to a 10 ml vial with a magnetic stirrer bar under argon atmosphere. The vial was then sealed with a cap and the mixture was stirred in an aluminium block at 23° C. during a time as indicated in table 2. The reaction mixture was then filtered to remove the catalyst and ligand. All volatiles (including non-reacted 2,5-dimethylfuran) were removed in vacuo at 50° C./<30 mbar to give an oily residue. The amounts of 2,4,6-TMP and 2,3,6-TMP were determined by GC (gas chromatography). The ratio as well as the yield are indicated in table 2.

TABLE 2
Reaction of 2,5-dimethylfuran with propyne in different solvents
and different catalysts

| | | | Ligand | Time | Yield | 2,4,6: |
|---|---|---|---|---|---|---|
| Ex. | Propyne¹ | Ligand | [mol %]² | [h] | [wt %]³ | 2,3,6⁴ |
| 27 | acetone | (PhO)₂P═O(OH) | 6 | 148 | 3 | 3:97 |
| 28 | acetone | P(Ph_{5F})₃ | 6 | 120 | 34 | 8:92 |
| 29 | acetone | P(Ph_{5F})₃ | 6 | 148 | 33 | 5:95 |
| 30 | 3-pentanone | P(Ph_{5F})₃ | 6 | 240 | 29 | 7:93 |
| 31 | 3-pentanone | P(Ph_{5F})₃ | 6 | 240 | 24 | 7:93 |
| 32 | 3-pentanone | (MeO)P(OPh)₂ | 6 | 148 | 3 | 3:97 |
| 33 | 3-pentanone | (PhO)₂P═O(OH) | 6 | 148 | 15 | 2:98 |
| 34 | 3-pentanone | P(OPh)₃ | 6 | 148 | 6 | 2:98 |
| 35 | 3-pentanone | P(Ph_{5F})₃ | 6 | 148 | 39 | 4:96 |
| 36 | 3-pentanone | P(Ph_{5F})₃ | 6 | 240 | 34 | 6:94 |

¹propyne 2% in mentioned organic solvent
²relative to 2,5-dimethylfuran
³combined yield of 2,4,6-TMP and 2,3,6-TMP
⁴2,4,6:2,3,6: ratio 2,4,6-TMP and 2,3,6-TMP (area %))

Third Experimental Series (Examples 37-43): Reaction of 2,5-Dimethylfuran with MAF

2,5-dimethylfuran was reacted with MAF gas (2% by weight) in an organic solvent and a platinum catalyst as indicated in table 2.

For this, the platinum chloride (6 mol %), 2,5-dimethylfuran (1.0 equivalent) and MAF gas (1.0 equivalent) dissolved (2% by weight) in the respective organic solvent were added to a 10 ml vial with a magnetic stirrer bar under argon atmosphere. The vial was then sealed with a cap and the mixture was stirred in an aluminium block at 23° C. during a time as indicated in table 3. The reaction mixture was then filtered to remove the catalyst and ligand. All volatiles (including non-reacted 2,5-dimethylfuran) were removed in vacuo at 50° C./<30 mbar to give an oily residue. The amounts of 2,4,6-TMP and 2,3,6-TMP were determined by GC (gas chromatography). The ratio as well as the yield are indicated in table 2.

Reaction of 2,5-dimethylfuran with propyne/propadiene (MAF) in
different solvents and different catalysts

| | | | Ligand | Time | Yield | 2,4,6: |
|---|---|---|---|---|---|---|
| Ex. | MAF¹ | Ligand | [mol %]² | [h] | [wt %]³ | 2,3,6⁴ |
| 37 | 3-pentanone | P(OPh)₃ | 6 | 148 | 4 | 1:99 |
| 38 | 3-pentanone | P(OPh)₃ | 12 | 148 | 8 | 1:99 |
| 39 | 3-pentanone | (MeO)P(OPh)₂ | 6 | 148 | 6 | 2:98 |
| 40 | 3-pentanone | (PhO)₂P═O(OH) | 6 | 148 | 13 | 1:99 |
| 41 | 3-pentanone | (PhO)₂P═O(OH) | 12 | 148 | 23 | 2:99 |
| 42 | acetone | P(Ph_{5F})₃ | 6 | 148 | 5 | 4:96 |
| 43 | 3-pentanone | P(Ph_{5F})₃ | 6 | 148 | 27 | 4:96 |

¹MAF 2% in mentioned organic solvent
²relative to 2,5-dimethylfuran
³combined yield of 2,4,6-TMP and 2,3,6-TMP
⁴2,4,6:2,3,6: ratio 2,4,6-TMP and 2,3,6-TMP (area %)

### Example 44: Methylation: Step a′)

A gas-phase flow reactor was filled with an iron oxide-based catalyst (13 g). The reactor was closed and heated under nitrogen to 350° C. in the reactor. The feed consisted of a mixture of 2,5-dimethyl phenol and 2,4-dimethyl phenol (example 2) and methanol and water in a molar ratio of 0.35:0.65:30:1.7. The mixture was pumped at 0.39 ml/min from top to bottom into the gas-phase reactor. After the heated zone the reaction mixture was cooled to room temperature and collected in a bottle. The bottle was emptied and analysed after each 24 h for five days.

The average conversion and selectivity over the five days were:
Conversion of 2,5-dimethyl phenol: 16%
Conversion of 2,4-dimethyl phenol: 32%
Selectivity to 2,3,6-trimethyl phenol: 95%
Selectivity to 2,4,6-trimethyl phenol: 89%

### Example 45: Photoxidation of Mixture of 2,4,6-TMP and 2,3,6-TMP: Step b)

A solution of mixture of 2,4,6-TMP (3.3 mmol) and 2,3,6-TMP (1.7 mmol)) and methylene blue hydrate ([CAS: 122965-43-9]),14.4 mg, 0.900 mol %) in methanol and water (4:1, v/v, 250 mL) was prepared to give a homogenous blue solution. The solution was pumped through a high-pressure liquid chromatography pump into the photoreactor (tubing system: 0.75 mm internal diameter, 1.58 mm outer diameter, PFA coil) (liquid flow rate: 0.250-0.023 mL/min, HPLC regulated piston pump) with a constant pressure of 10 bar.

Before entering the photoreactor, the solution was enriched with air (air flow rate: 1.350-0.125 mL/min, mass flow controller. Inside the photoreactor, the reaction mixture was exposed to a hyper red LED light source during a residence time of 40 min. Complete conversion was confirmed by thin layer chromatography and by QNMR. The photoreactor was kept at ambient temperature (20° C.). The reaction mixture (25 mL) was collected after two residence times by a round bottom. Water (50 mL) was added and the solution was extracted with pentane (2×50 mL) and diethyl ether (2×50 mL). The combined organic layers were washed with brine (50 mL), dried over Na₂SO₄, filtered and the organic solvent was removed under reduced pressure (15 mbar) to yield a mixture of 4-hydroperoxy-2,4,6-trimethyl-cyclohexa-2,5-dien-1-one)(formula (IIIa)) (conversion: >99%, yield: 99%) and 2,3,5-trimethylbenzoquinone (=2,3,5-trimethylcyclohexa-2,5-diene-1,4-dione, TMQ) (formula (IIIb)) (conversion: >99%, yield: 71%).

### Example 46: Reduction of Mixture of 4-hydroperoxy-2,4,6-trimethylcyclohexa-2,5-dien-1-one) and 2,3,5-trimethyl,benzo,quinone: Step c)

Platinum on charcoal (1.0 mol %) was charged in a flame-dried flask and methanol was carefully added to give a dark heterogenous solution. A solution of mixture of of 4-hydroperoxy-2,4,6-trimethylcyclohexa-2,5-dien-1-one)(formula (IIIa)) and 2,3,5-trimethylbenzoquinone (=2,3,5-trimethylcyclohexa-2,5-diene-1,4-dione, TMQ) (formula (IIIb)) (0.25 mmol) in methanol and water (4:1, v/v) is prepared to give a yellow homogeneous solution which was given to the catalyst solution. The flask was evacuated and then hydrogen was introduced. The reaction mixture was stirred for >1 hour at ambient temperature (20° C.) until complete conversion. Water (10 mL) was added and the solution is extracted with diethyl ether (3×5 mL). The combined organic layers were dried over Na₂SO₄, filtered and the organic solvent is removed under reduced pressure (15 mbar) to yield a mixture of 2,3,5-trimethylhydroquinone (formula (I)) (conversion: >99%, yield: 74%)) and 4-hydroxy-2,4,6-trimethylcyclohexa-2,5-dien-1-one (formula (IV)) (conversion: >99%, yield: 51%)).

### Example 47: Rearrangement of 4-hydroxy-2,4,6-trimethylcyclohexa-2,5-dien-1-one to 2,3,5-trimethylhydroquinone: Step d)

The mixture of 2,3,5-trimethylhydroquinone (formula (I)) and 4-hydroxy-2,4,6-trimethylcyclohexa-2,5-dien-1-one (formula (IV)) in an aqueous NaOH solution and methanol and sodium sulphite (6.3 mol-%, relative to formula (IV), was pumped through a flow reactor (diameter 1.5 mm, length: 2000 mm) with 10 ml/min at 250° C. The solution was neutralized at the end of the flow reactor with sulfuric acid. The reaction mixture was extracted with ethyl acetate, dried over MgSO₄, and concentrated in vacuo. 2,3,5-trimethylbenzoquinone (formula (I)) was obtained in 92% yield in total.

1. A process for manufacturing 2,3,5-trimethylhydroquinone of the formula (I) 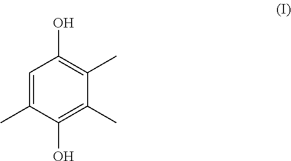
comprising the consecutive steps
a) providing a mixture of the compound of the formula (IIa) and the compound of the formula (IIb), 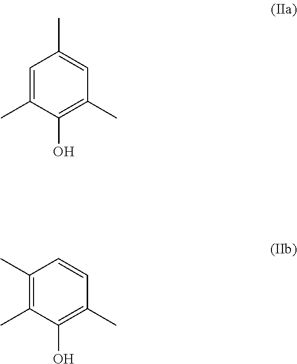
b) oxidizing the mixture of formula (IIa) and the compound of the formula (IIb) to form a mixture of the compound of the formula (IIIa) and of the compound of the formula (IIIb), 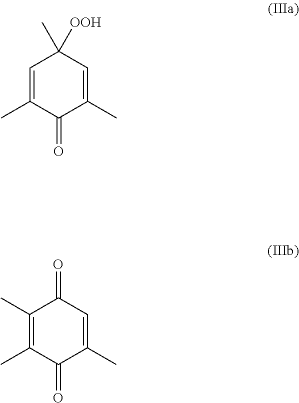
c) reducing the mixture of the compound of the formula (IIIa) and of the compound of the formula (IIIb) by means of a reduction agent to yield a mixture of the compound of the formula (IV) and of the compound of the formula (I), 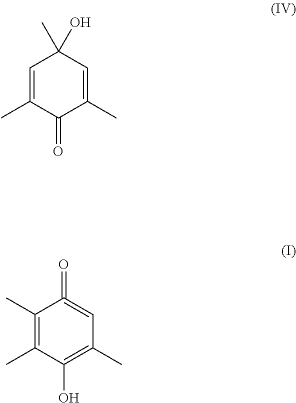
d) rearranging the compound of the formula (IV) in a mixture of the compound of the formula (IV) and of the compound of the formula (I) by treatment of said mixture with a basic substance at a temperature of >200° C., preferably >240° C., to yield the compound of the formula (I)

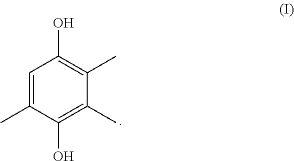

2. The process according to claim 1, characterized in that the mixture of the compound (IIa) and the compound of the formula (IIb) is obtained by a reaction step a0) a0) reacting the compound of the formula (V) with the compound of the formula (VI) 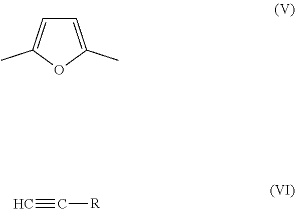
wherein R represents H or CH₃, preferably CH₃;
in the presence of a Pt or an Au catalyst, which is either in the form a salt or of a complex to form a mixture of the compound of the formula (IIa-1) and of the formula (II-b) 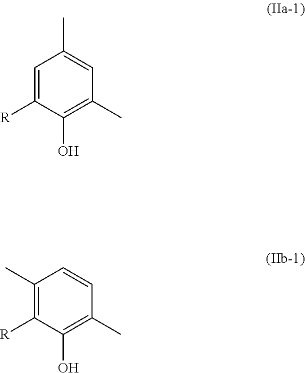
with the proviso that in case R represents H, the mixture of the compound of the formula (IIa-1H) and of the formula (IIb-1H) is submitted to a reaction step a′) 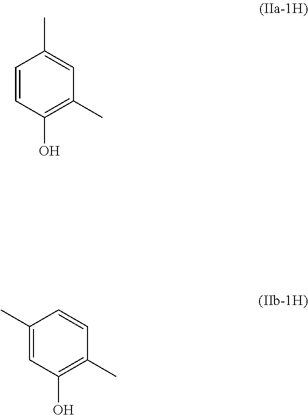
a′) methylation of the mixture of the formula (IIa-1H) and of the formula (IIb-1H) to yield the mixture of the compound of the formula (IIa) and of the formula (IIb)

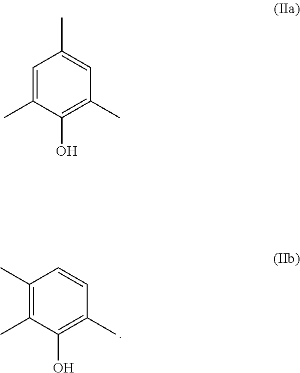

3. The process according to claim 2, characterized in that the mixture of the step a0) is performed in in the presence of a Pt catalyst, which is either in the form a Pt salt or of a Pt complex.

4. The process according to claim 2 characterized in that in case R represents CH₃, propyne is used in said reaction in combination with propadiene.

5. The process according to claim 2, characterized in that the Pt catalyst is a Pt(II) salt, particularly PtCl₂.

6. The process according to claim 2, characterized in that the Pt catalyst is a complex of Pt(II) having at least one organic ligand comprising at least one phosphorous atom, particularly a ligand selected from the group consisting of phosphites, phosphates, phosphonates, and phosphines.

7. The process according to claim 2, characterized in that the Pt catalyst is PtCl₂ in the presence of an organic ligand having at least comprising at least one phosphorous atom, particularly a ligand selected from the group consisting of phosphites, phosphates, phosphonates, and phosphines.

8. The process according to claim 6, characterized in that the organic ligand is selected from the group consisting of triphenylphosphite (P(OPh)₃), diphenylphosphate ((PhO)₂P═O(OH)), triphenylphosphate ((PhO)₃P═O), triethylphosphate ((EtO)₃P═O), dibenzylphosphite ((PhCH₂O)₂P═O(H)), methyldiphenylphosphite ((MeO)P(OPh)₂), triphenylphosphine (P(Ph)₃), P(Ph_{5F})₃, bis-(2,2,2-trifluorethyl)-phosphite ((CF₃CH₂O)₂P═O(H)), triethyl 2-fluoro-2-phosphonoacetate ((CHFCOOEt)(EtO)₂P═O), tris(2-tolyl)phosphite, ethyl 3-(diethoxyphosphoryl)propanoate (=triethyl 3-phosphonopropionate) ((CH₂CH₂COOEt)(EtO)₂P═O), 2-(diphenylphosphino)benzoic acid (P(Ph)₂C₆H₄COOH), 3,9-Bis(2,4-di-tert-butylphenoxy)-2,4,8,10-tetraoxa-3,9-diphosphaspiro[5.5]undecane, P(O-isooctyl)₃), P(O-isopropyl)₃), P(Oi-PrF₆)₃, P(On-Bu)₃, bis(2,4-di-tert-butylphenoxy)-1²-phosphane, CH₃P(EtO)₂, 1,1′-binaphthyl-2,2′-diyl hydrogenphosphate (=4-hydroxydinaphtho[2,1-d:1′,2′-][1,3,2]dioxaphosphepine 4-oxide), VAPOL hydrogen phosphate (=18-hydroxy-8,9-diphenyldiphenanthro[4,3-d:3′,4′-J]1[1,3,2]dioxaphosphepine 18-oxide), methyltriphenoxyphosphonium iodide ([CH₃P(OPh)₃]⁺I⁻, 4-ethyl-2,6,7-trioxa-1-phosphabicyclo[2.2.2]octane ((CH₂CH₂COOEt)(EtO)₂P═O, trimethylolpropane phosphite), trisodium triphenylphosphine trisulfonate (=TPPTS), disodium triphenylphosphinedisulfonate (=TPPDS) and sodium triphenylphosphinemonosulfonate (=TPPMS);
wherein Ph represents phenyl, Ph_{5F} represents pentafluorophenyl and Et represents ethyl and Me represents methyl.

9. The process according to claim 1, characterized in that the mixture of the compound (IIa) and the compound of the formula (IIb) is obtained by the reaction step a″)
a″) methylation of a mixture of p-cresol of the formula (0p) and m-cresol of the formula (0m) 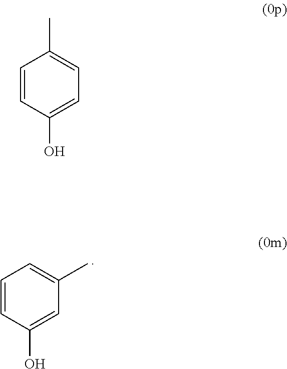
to yield the mixture of the compound of the formula (IIa) and of the formula (IIb).

10. The process according to claim 1 characterized in that the oxidation step b) is a photooxidation using oxygen and a photosensitizer of the formula (X) 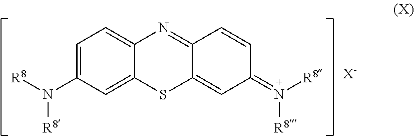
wherein R⁸, R^{8′}, R^{8″} and R^{8′″} independently from each other represent either a H, or a C₁₋₄ alkyl group;
or
wherein R⁸ and R^{8′} and/or R^{8″} and R^{8′″} form together with N a five or six membered ring;
with the proviso that at least one of the residues R⁸, R^{8′}, R^{8″} and R^{8′″} is different from H;
and X⁻ represents an anion;
in a solvent mixture of water and at least one C₁₋₈ alkanol or at least one C₂₋₄ alkylene diol;
and using light which has a peak wavelength (λₘₐₓ) in its spectrum in the range of between 580 and 780 nm.

11. The process according to claim 10 characterized in that used light has a peak wavelength (λₘₐₓ) in its spectrum in the range of between 625 and 740 nm.

12. The process according to claim 10, characterized in that the light source for the light is a white LED lamp in combination with a filter blocking wavelengths below 500 nm, particularly below 625 nm.

13. The process according to claim 10, characterized in that the light source for the light is a red LED lamp.

14. The process according to claim 10 characterized in that the solvent mixture is a mixture of water and methanol and/or ethanol and/or isopropanol.

15. The process according to claim 10, characterized in that R⁸═R^{8′}═R^{8″}═R^{8′″}═CH₃ and preferably X⁻ represents a halide, particularly chloride.

16. The process according to claim 1 characterized in that the reducing step c) is performed in the presence of a reducing agent which is selected from the group consisting of thiosulphates, tertiary phosphine, hydrogen, dithionates, dithionites, sulfites, trialkylphosphites, iodides, metals and dialkylsulfides.

17. A process of manufacturing α-tocopherol comprising the steps
i) providing 2,3,5-trimethylhydroquinone manufactured from a mixture of mesitol (formula (IIa)) and 2,3,6-TMP (formula (IIb)) by a process according to claim 1; 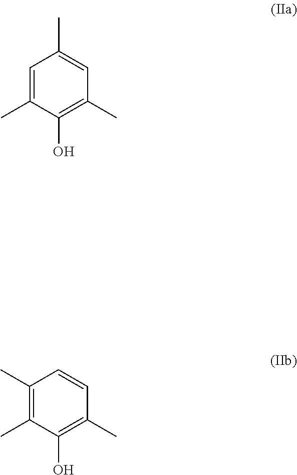
ii) condensing 2,3,5-trimethylhydroquinone from step i) with a compound of the formula (VII-A) or of the formula (VII-B) to yield α-tocopherol of the formula (VIII) 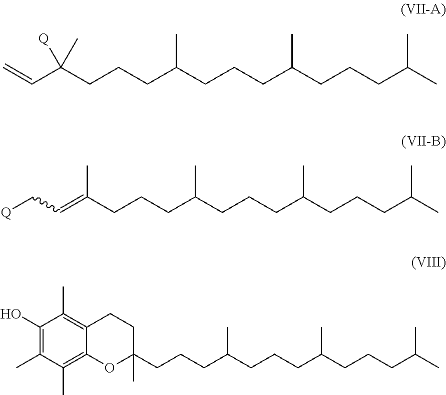
wherein Q is a substituent selected from the group consisting of OH, halide,
acyloxy, particularly acetoxy or benzoyloxy, methanesulfonyloxy (=mesyloxy), ethanesulfonyloxy, benzenesulfonyloxy and toluenesulfonyloxy (=tosyloxy); and
wherein any wavy line represents a carbon-carbon bond which when linked to the carbon-carbon double bond is either in the Z- or in the E-configuration.
